(19) United States
(12) Patent Application Publication
(10) Pub. No.: US 2023/0132735 A1
(43) Pub. Date: May 4, 2023

(54) METHOD AND APPARATUS FOR PREPARATION OF ALKYLENE CARBONATE USING POLYAMINE-HETEROGENEOUS CATALYST

(71) Applicant: KOREA INSTITUTE OF SCIENCE AND TECHNOLOGY, Seoul (KR)

(72) Inventors: In Wook NAH, Seoul (KR); Jae Ik KIM, Seoul (KR); Soon Jong KWAK, Seoul (KR); In Hwan OH, Seoul (KR)

(21) Appl. No.: 18/049,085

(22) Filed: Oct. 24, 2022

(30) Foreign Application Priority Data
Nov. 2, 2021 (KR) 10-2021-0149170

(51) Int. Cl. C07D 317/36; B01J 27/24

(52) U.S. Cl. CPC C07D 317/36; B01J 27/24

(57) ABSTRACT

Disclosed are a method and an apparatus for preparing an alkylene carbonate using a polyamine-based heterogeneous catalyst. The rapid increase in reactor temperature and the risk of explosion due to heat of reaction may be prevented by recycling the produced alkylene carbonate to the reactor. Accordingly, it is possible to obtain the alkylene carbonate stably and continuously with high yield while maintaining reaction temperature and pressure constant.

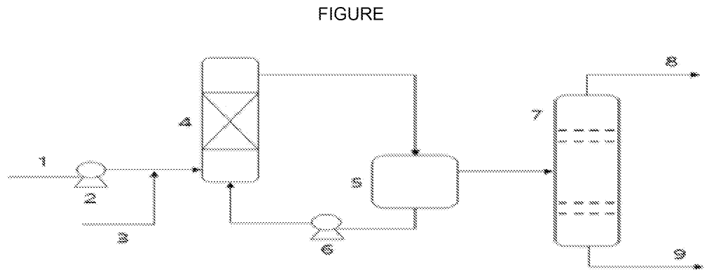

### CROSS-REFERENCE TO RELATED APPLICATION

This application claims priority to Korean Patent Application No. 10-2021-0149170, filed on Nov. 2, 2021, and all the benefits accruing therefrom under 35 U.S.C. § 119, the contents of which in its entirety are herein incorporated by reference.

### BACKGROUND

### 1. Field

The present disclosure relates to a method and an apparatus for continuously preparing an alkylene carbonate using a polyamine-based heterogeneous catalyst.

Korean Patent Registration No. 1804762 and Korean Patent Application Publication No. 2020-21319 filed by the inventors of the present disclosure are incorporated herein in their entireties.

### Description about National Support Research and Development

This research is conducted by Korea Institute of Science and Technology under the support of Ministry of Land, Infrastructure and Transport of Korea, and the subject name thereof is Discovery and development of greenhouse gas reduction-absorption-resource conversion technology applicable to urban facilities and infrastructure (Subject Identification No.: 1615012190).

### 2. Description of the Related Art

Alkylene carbonates such as ethylene carbonate, propylene carbonate, etc. are widely used in industrial processes as solvents and diluents. They are also used as cosmetic ingredients and secondary battery electrolytes. In addition, because they can be used as intermediates when preparing alkylene glycols from alkylene oxides, the interest in alkylene carbonates is increasing recently.

Formerly, alkylene carbonates were prepared by reacting ethylene glycol with phosgene (COCl₂). This reaction is performed at room temperature in the absence of a catalyst. However, due to the fatal toxicity of the phosgene and the formation of hydrogen chloride as a byproduct during the process, which pollutes the environment, a process of reacting alkylene oxides with carbon dioxide is mainly used recently.

However, the process of preparing alkylene carbonates by reacting alkylene oxides with carbon dioxide requires high temperature and high pressure, unlike the phosgene process occurring at room temperature in the absence of a catalyst, a large quantity of byproducts may be generated from the decomposition or polymerization of the alkylene oxides and there is a risk of explosion.

In order to solve these problems, researches have been carried out to develop various catalysts to reduce cost and improve efficiency.

For example, Japanese Patent Publication No. H9-67365 describes a method of using KI as a catalyst, and Japanese Patent Publication No. S59-13776 describes a method of using tributylmethylphosphonium iodide and a tetraalkylphosphonium halide.

In addition, Japanese Patent Publication No. H9-235252 discloses a method of using a polystyrene copolymer having a quaternary phosphonium halide as its terminal group. This document describes that a yield of 50-95% is achieved when reaction is performed at 100-170° C. for 1-5 hours.

However, these catalysts are very expensive ionic liquids. Actually, the commercialized process of using an inorganic halide catalyst as a homogeneous catalyst requires a reaction temperature of 180° C., a reaction pressure of 100 atm and a reaction time of 8 hours or longer, and the water content in the carbon dioxide and the alkylene oxide should be controlled to a few hundred ppms or lower.

In addition, Japanese Patent Publication No. H7-206846 discloses a method of using an ion-exchange resin substituted with catalysts such as CsOH, RbOH and ammonium halides.

In addition, U.S. Pat. No. 4,233,221 describes a method of using Dowex and Amberlite ion-exchange resins. However, the yield of alkylene carbonate of this method is low as merely about 30-80%.

In addition to the above-mentioned catalysts, U.S. Pat. No. 5,283,356 discloses a method of using a phthalocyanine complex containing Co, Cr, Fe, Mn, Ni, Ti, V, Zr, etc. as a catalyst, and Japanese Patent Publication No. H7-206547 presents a method of using a catalyst wherein the hydrogen ion of a heteropoly acid is substituted by a rubidium (Rb) or cesium (Cs) ion. However, the two cases require expensive catalysts and the yield is low as 30-90% although the reaction temperature is relatively mild at 120-180° C.

As described above, the large-scale production of an alkylene carbonate by the methods according to the prior art is disadvantageous in that reaction conditions are complicated such as high temperature and pressure, long reaction time, difficulty in removal of water, etc. and selectivity and yield at high temperature are low.

In addition, because most of the catalysts of the prior art have poor thermal stability, they tend to be decomposed partially during reaction, distillation or purification at high temperature to produce halide ions. The produced halide ions react with alkylene oxides to produce halide-based byproducts, or the prepared alkylene carbonates are even decomposed back to carbon dioxide and alkylene oxides.

The inventors of the present disclosure have proposed a polyamine-containing catalyst for preparing a heterogeneous alkylene carbonate, a method for preparing the same and a method and an apparatus for preparing an alkylene carbonate using the catalyst in order to prepare an alkylene carbonate in short time and with high yield under a mild reaction condition of low temperature and low pressure by facilitating the reaction between an alkylene oxide and carbon dioxide (Korean Patent Registration No. 1804762 and Korean Patent Application Publication No. 2020-21319).

Since a commercialized process of preparing an alkylene carbonate requires continuous preparation using a homogeneous catalyst, it is necessary to develop a method and an apparatus suitable for continuous preparation using the polyamine-based heterogeneous catalyst.

### SUMMARY

In an aspect, the present disclosure is directed to providing a method and an apparatus that allow the continuous preparation of an alkylene carbonate using a polyamine-based heterogeneous catalyst while maintaining stable and high yield at low temperature and low pressure.

The present disclosure provides a method for preparing an alkylene carbonate, which includes a step of reacting an alkylene oxide in liquid state and carbon dioxide in gas state in a catalytic reactor with a polyamine-based catalyst in solid state via upward flow.

In an exemplary embodiment, at least a part of the produced alkylene carbonate may be recycled to the catalytic reactor so that the unreacted carbon dioxide and alkylene oxide dissolved in the produced alkylene carbonate are provided again to the reaction solution.

In an exemplary embodiment, the alkylene carbonate may be prepared continuously while the temperature of the catalytic reactor is maintained constant.

In an exemplary embodiment, a recycle ratio defined as the ratio of the recycled volume to the volume of the introduced alkylene oxide may be in a range from 5 to 100 or from 20 to 60.

In an exemplary embodiment, the reaction may be performed at a temperature of 100-170° C. and a pressure of 10-40 atm for 1-8 hours.

In an exemplary embodiment, the catalyst may be one or more catalysts of Chemical Formulas 1 and 2.

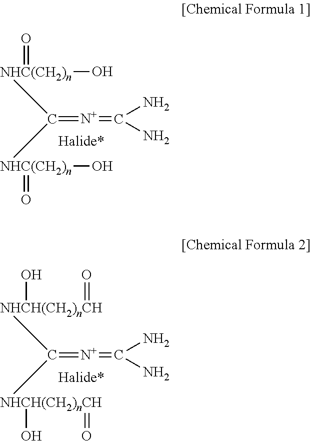

In [Chemical Formula 1] and [Chemical Formula 2], n is a repeat unit, which is an integer from 1 to 30.

The present disclosure also provides an apparatus for preparing an alkylene carbonate, which includes: a reactor filled with a polyamine-based heterogeneous catalyst; a pump which supplies an alkylene oxide and carbon dioxide as raw materials to the reactor; a storage tank wherein an alkylene carbonate produced in the reactor is stored; and a pump which recycles at least a part of the produced alkylene carbonate to the reactor.

In an exemplary embodiment, the apparatus may further include a distillation tower connected to the storage tank and, when the alkylene carbonate in the storage tank reaches a predetermined level, the alkylene carbonate above the predetermined level may be supplied to the distillation tower.

According to the method for preparing an alkylene carbonate using a polyamine-based heterogeneous catalyst of the present disclosure, the rapid increase in reactor temperature and the risk of explosion due to heat of reaction may be prevented by recycling the produced alkylene carbonate to the reactor. Accordingly, it is possible to obtain the alkylene carbonate stably and continuously with high yield while maintaining reaction temperature and pressure constant.

### BRIEF DESCRIPTION OF THE DRAWINGS

FIGURE schematically shows an apparatus for continuously preparing an alkylene carbonate using a polyamine-based heterogeneous catalyst according to the present disclosure.

### DETAILED DESCRIPTION

### Definition of Terms

In the present specification, channeling refers to the phenomenon in which, when a reactant passes through a catalyst layer, the reactant flows along only one side of the catalyst layer.

### Description of Exemplary Embodiments

Hereinafter, exemplary embodiments of the present disclosure are described in detail.

The preparation of an alkylene carbonate is an exothermic reaction, and raw materials such as ethylene oxide or propylene oxide are explosive compounds. The present disclosure provides a method for continuous, non-batch-type preparation which maximizes reaction yield while safely maintaining low temperature and low pressure to prevent explosion.

Specifically, a method for preparing an alkylene carbonate according to the present disclosure includes a step of reacting an alkylene oxide in liquid state and carbon dioxide in gas state in a catalytic reactor with a polyamine-based catalyst in solid state via upward flow. Through this, catalyst channeling can be prevented.

In an exemplary embodiment, the prepared alkylene carbonate may be recycled using a pressure pump so that the unreacted carbon dioxide and alkylene oxide dissolved in the alkylene carbonate can be introduced again into the reaction solution and react again with the polyamine-based heterogeneous catalyst layer.

In addition, the rapid increase of reactor temperature due to heat of reaction can be prevented by the alkylene carbonate. Accordingly, the alkylene carbonate can be prepared continuously while maintaining the reactor temperature and pressure constant.

In an exemplary embodiment, a recycle ratio defined as the ratio of the recycled volume to the volume of the introduced alkylene oxide may be in a range from 5 to 100 or from 20 to 60.

In an exemplary embodiment, the method for preparing an alkylene carbonate according to the present disclosure may be performed by batch or continuous operation, specifically by continuous operation.

In an exemplary embodiment, the catalyst may be one or more catalyst represented by [Chemical Formula 1] or [Chemical Formula 2].

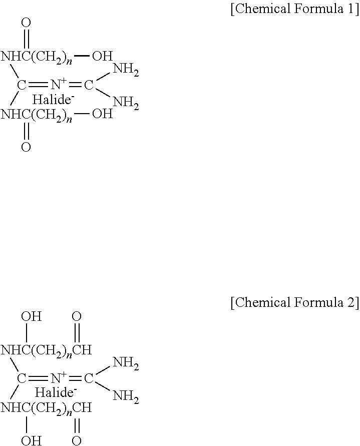

In [Chemical Formula 1] and [Chemical Formula 2], n is a repeat unit, which is an integer, e.g., from 1 to 30.

In an exemplary embodiment, the alkylene oxide may be a compound represented by [Chemical Formula 3], and the alkylene carbonate may be a compound represented by [Chemical Formula 4].

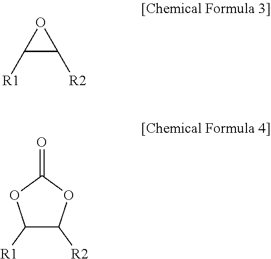

In [Chemical Formula 3] and [Chemical Formula 4], each of R1 and R2 may be independently hydrogen, a C₁₋₆ alkyl group, a C₁₋₆ haloalkyl group or an aryl group, and may form a hexagonal ring together with the carbon atoms to which they are bound.

In an exemplary embodiment, the alkylene oxide represented by [Chemical Formula 3] includes, for example, ethylene oxide, propylene oxide, epichlorohydrin, butylene oxide, styrene oxide, cyclohexylene oxide, etc., although not being limited thereto.

In an exemplary embodiment, the reaction may be performed at a temperature of 100-170° C. and a pressure of 10-40 atm for 1-8 hours. If the reaction temperature is below 100 ° C., the rate of reaction is slow, and side reactions occur at temperatures above 170° C. If the reaction pressure is below 10 atm, the reaction does not proceed well. And, if the reaction pressure is above 40 atm, it is undesirable in terms of economy because the increase in efficiency is insignificant. If the reaction time is shorter than 1 hour, the reaction does not proceed well. And, if the reaction time is longer than 8 hours, the economic efficiency is decreased.

In the prior art, the reaction of an alkylene oxide and carbon dioxide for preparation of an alkylene carbonate has been conducted at a temperature of 100-180° C. and a pressure of 10-100 atm. The inventors of the present disclosure have shown in Korean Patent Registration No. 1804762 and Korean Patent Application Publication No. 2020-21319 that high yield can be achieved, for example, at 150° C. and 20 atm by using a polyamine-based heterogeneous catalyst.

In exemplary embodiment, the present disclosure provides an apparatus for preparing an alkylene carbonate, which includes: a reactor filled with a polyamine-based heterogeneous catalyst; a pump which supplies an alkylene oxide and carbon dioxide as raw materials to the reactor; a storage tank wherein an alkylene carbonate produced in the reactor is stored; and a pump which recycles at least a part of the produced alkylene carbonate to the reactor.

FIGURE schematically shows an apparatus for continuously preparing an alkylene carbonate using a polyamine-based heterogeneous catalyst according to the present disclosure.

As can be seen from FIGURE, in an exemplary embodiment of the present disclosure, an alkylene oxide 1, which is a raw material, is introduced to a reactor 4 in which a polyamine-based heterogeneous catalyst is filled by a pressure pump 2 quantitatively via upward flow together with carbon dioxide 3.

An alkylene carbonate produced in the reactor 4 by catalytic reaction is transferred to a storage tank 5. Some of the alkylene carbonate is recycled to the reactor 4 by a pressure pump 6. When the storage tank 5 reaches a predetermined level, the alkylene carbonate above the predetermined level is discharged to a distillation tower 7 for separation and purification.

As described above, a recycle ratio (recycled volume of alkylene oxide (mL/min)/volume of introduced alkylene oxide (mL/min)) may be in a range from 5 to 100. If the recycle ratio is below 5, the reactor temperature may be increased excessively due to the decreased cooling effect of the catalytic reactor. And, if the recycle ratio is above 100, operation cost may be increased. Accordingly, a recycled volume of 5-100 is preferred for the preparation.

Hereinafter, the present disclosure is described in more detail through examples. However, the present disclosure is not limited by the examples. It should be understood that the present disclosure may be embodied in various forms included in the scope of the present disclosure defined in the appended claims and the following examples are provided such that the present disclosure is more complete and can be easily carried out by those having ordinary knowledge in the art.

### Apparatus Configuration

Continuous reaction was conducted using the apparatus shown in FIGURE. The maximum capacity of an alkylene oxide injection pump was 10 mL/min, and the reactor 4 was a vertical tubular reactor with a diameter of 2 inches and a total volume of 0.65 L. The circulation pump 6 was a flow volume-controllable metering pump with a maximum capacity of 100 mL/min, and the storage tank 5 was a vertical tubular reactor with a diameter of 6 inches and a total volume of 6 L. The flow rate of carbon dioxide was controlled with pressure and a flow rate controller.

### EXAMPLE 1

After filling 120 g of a polyamine-based heterogeneous catalyst of Chemical Formula 1 (grain size: 2 mm or larger) and supplying 2 kg of propylene carbonate into a reactor, the reactor temperature was raised to 150° C.

The amount of the initially supplied propylene carbonate solvent was set to 30 mL/min and the supply amount of propylene oxide was set to 1 mL/min using a circulation pump in consideration amount of the production amount. In addition, carbon dioxide was supplied using a pressure regulating valve so that the reaction pressure was maintained at 20 atm. The reaction temperature showed a distribution from 107° C. up to 156° C. depending on the height of the catalyst layer in the reactor.

After conducting the propylene carbonate production reaction for 4 hours under the conditions described above, the supply of propylene oxide was stopped and the reaction temperature was maintained until the consumption of carbon dioxide was stopped. The purity of the produced propylene carbonate was 99% or higher and the reaction yield was 95.5%. 99.5% propylene carbonate could be obtained by conducting simple distillation at 122° C. and 0.1 Torr.

### EXAMPLE 2

After filling 120 g of a polyamine-based heterogeneous catalyst of Chemical Formula 2 (grain size: 2 mm or larger) and supplying 2 kg of ethylene carbonate into a reactor, the reactor temperature was raised to 150° C.

The amount of the initially supplied ethylene carbonate solvent was set to 30 mL/min and the supply amount of ethylene oxide was set to 1 mL/min using a circulation pump in consideration amount of the production amount. In addition, carbon dioxide was supplied using a pressure regulating valve so that the reaction pressure was maintained at 20 atm. The reaction temperature showed a distribution from 110° C. up to 154° C. depending on the height of the catalyst layer in the reactor.

After conducting the ethylene carbonate production reaction for 4 hours under the conditions described above, the supply of ethylene oxide was stopped and the reaction temperature was maintained until the consumption of carbon dioxide was stopped. The purity of the produced ethylene carbonate was 99% or higher and the reaction yield was 96.0%. 99.6% ethylene carbonate could be obtained by conducting simple distillation at 122° C. and 0.1 Torr.

### EXAMPLES 3-7

Propylene carbonate production reaction was conducted in the same manner as in Example 1 while varying the reaction pressure and the recycling amount of propylene carbonate. The reaction conditions and results are described in Table 1.

TABLE 1

| | | | | Recycle | |
|---|---|---|---|---|---|
| | | | | ratio | |
| | | | Inflow | (recycled | |
| | Reaction | Reaction | volume | volume/ | Reaction |
| | pressure | temperature | (mL/ | inflow | yield |
| Example | (atm) | (° C.) | min) | volume) | (%) |
| 3 | 10 | 152 | 1 | 40 | 85.0 |
| 4 | 20 | 151 | 1 | 40 | 92.7 |
| 5 | 20 | 156 | 1 | 30 | 95.5 |
| 6 | 20 | 143 | 1 | 40 | 87.3 |
| 7 | 20 | 151 | 0.5 | 35 | 98.6 |

It was confirmed that alkylene carbonates could be obtained continuously and stably with high yield according to the present disclosure.

### Detailed Description of Main Elements

1: alkylene oxide supply tube
2: pressure pump
3: carbon dioxide supply tube
4: catalytic reactor
5: alkylene carbonate storage tank
6: pressure pump
7: alkylene carbonate distillation device
8: alkylene carbonate recovery tube
9: high-melting-point impurity recovery tube

What is claimed is:

1. A method for preparing an alkylene carbonate, comprising a step of reacting an alkylene oxide in liquid state and carbon dioxide in gas state in a catalytic reactor with a polyamine-based catalyst in solid state via upward flow.

2. The method for preparing an alkylene carbonate according to claim 1, wherein at least a part of the produced alkylene carbonate is recycled to the catalytic reactor so that the unreacted carbon dioxide and alkylene oxide dissolved in the produced alkylene carbonate are provided again to the reaction solution.

3. The method for preparing an alkylene carbonate according to claim 1, wherein the alkylene carbonate is prepared continuously while the temperature of the catalytic reactor is maintained constant.

4. The method for preparing an alkylene carbonate according to claim 2, wherein a recycle ratio defined as the ratio of the recycled volume to the volume of the introduced alkylene oxide is in a range from 5 to 100.

5. The method for preparing an alkylene carbonate according to claim 3, wherein the reaction is performed at a temperature of 100-170° C. and a pressure of 10-40 atm for 1-8 hours.

6. The method for preparing an alkylene carbonate according to claim 1, wherein the catalyst is one or more catalyst of Chemical Formulas 1 and 2: 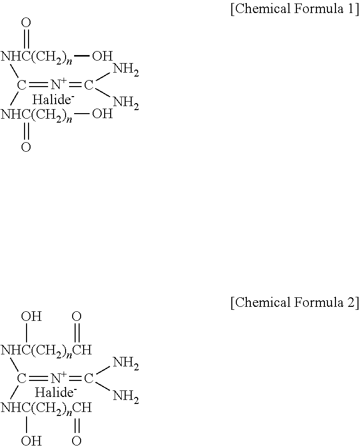
wherein n is a repeat unit, which is an integer from 1 to 30.

7. An apparatus for preparing an alkylene carbonate, comprising:
a reactor filled with a polyamine-based heterogeneous catalyst;
a pump which supplies an alkylene oxide and carbon dioxide as raw materials to the reactor;
a storage tank wherein an alkylene carbonate produced in the reactor is stored; and
a pump which recycles at least a part of the produced alkylene carbonate to the reactor.

8. The apparatus for preparing an alkylene carbonate according to claim 7, wherein the alkylene carbonate is prepared continuously while the temperature of the catalytic reactor is maintained constant.

9. The apparatus for preparing an alkylene carbonate according to claim 7, wherein a recycle ratio defined as the ratio of the recycled volume to the volume of the introduced alkylene oxide is in a range from 5 to 100.

10. The apparatus for preparing an alkylene carbonate according to claim 8, wherein a reaction is performed at a temperature of 100-170° C. and a pressure of 10-40 atm for 1-8 hours.

11. The apparatus for preparing an alkylene carbonate according to claim 7, which further comprises a distillation tower connected to the storage tank and, when the alkylene carbonate in the storage tank reaches a predetermined level, the alkylene carbonate above the predetermined level is supplied to the distillation tower.

12. The apparatus for preparing an alkylene carbonate according to claim 7, wherein the catalyst is one or more catalyst of Chemical Formulas 1 and 2: 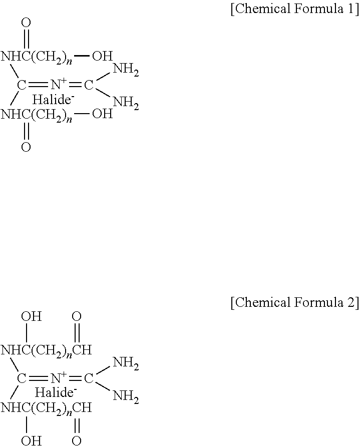
wherein n is a repeat unit, which is an integer from 1 to 30.
